(19) United States
(12) Patent Application Publication
(10) Pub. No.: US 2025/0114292 A1
(43) Pub. Date: Apr. 10, 2025

(54) METHOD AND SYSTEM FOR REMOVING MAKEUP

(71) Applicant: L’ORÉAL, Paris (FR)

(72) Inventors: Christopher Pang, Nanuet, NY (US); Tianyi Liu, Springfield, PA (US)

(73) Assignee: L’ORÉAL, Paris (FR)

(21) Appl. No.: 18/984,099

(22) Filed: Dec. 17, 2024

Related U.S. Application Data
(62) Division of application No. 17/144,533, filed on Jan. 8, 2021.

(51) Int. Cl. A61K 8/891; A61K 8/04; A61K 8/81; A61Q 1/10; A61Q 1/14

(52) U.S. Cl. CPC A61K 8/891; A45D 40/0068; A61K 8/042; A61K 8/31; A61K 8/8117; A61K 8/92; A61Q 1/10; A61Q 1/14; A45D 2200/25; A61K 2800/30; A61K 2800/31; A61K 2800/34; A61K 2800/43; A61K 2800/87; A61K 2800/884

(57) ABSTRACT

A method and system or kit is provided, where makeup containing an alkane and a styrenic block copolymer can be easily removed if a separate makeup-removing composition that includes an alkane and a low viscosity dimethicone (having a viscosity of 60,000 cst or less) is applied. The dimethicone in this viscosity range causes the styrenic block copolymer to gel. After allowing a short period of time for the gelling to occur, the gelled copolymer and the remainder of the makeup can then be readily removed.

### CROSS-REFERENCE TO RELATED APPLICATIONS

The present application claims priority to U.S. patent application Ser. No. 17/144,533, filed Jan. 8, 2021, the contents of which are incorporated by reference herein in its entirety.

### TECHNICAL FIELD

The present invention relates to techniques for removing eye makeup, and specifically to a method and system and/or kit for removing an anhydrous makeup composition (such as a mascara) containing a styrenic block copolymer and an alkane solvent using a remover containing dimethicone with viscosities up to 60,000 cst and an alkane solvent.

### BACKGROUND

Cosmetic makeup is generally intended to be worn for long periods of time, and be resistant to rubbing off. However, in doing so, the makeup becomes harder to remove when the user wishes to remove it. Indeed, some makeup, especially those using styrenic block copolymers, can be extremely challenging to remove without using, e.g., harsh chemicals that may not be suited for use, e.g., near a user's eyes, or without significant effort on the user's part.

As such, a technique for easily removing makeup containing styrenic block copolymers is useful and desirable.

### BRIEF SUMMARY

It is disclosed that a makeup containing styrenic block copolymers can be easily removed if a separate composition that includes a low viscosity dimethicone (having a viscosity of 60,000 cst or less) is applied, which causes the styrenic block copolymer to gel, if alkane solvents are present in both compositions. The gelled copolymer and the remainder of the makeup can then be readily removed.

Thus, a first aspect of the present disclosure is drawn to a system or kit, that comprises, consists essentially of, or consists of the novel combination of specific makeup (e.g., mascara) compositions and specific makeup-removing compositions. The makeup composition must contain a styrenic block copolymer and an alkane. The makeup-removing composition must contain an alkane and one or more low viscosity dimethicones (each having a viscosity of 60,000 cst or less).

The alkane in the makeup composition and the alkane in the makeup-removing composition may be the same alkane or may be different alkanes. Optionally, each of the alkanes is an alkane having between 12 and 18 carbons. Optionally, the alkane in the makeup composition present in an amount of between 10% and 70% by weight of the mascara composition. Optionally, the alkane in the makeup-removing composition is present in an amount of between 30% and 90% by weight of the makeup-removing composition.

The styrenic block copolymer (such as hydrogenated styrene/butadiene copolymer) is preferably present in an amount of between 0.2% and 7.0% by weight of the makeup composition.

The dimethicone is preferably present in an amount of between 10% and 70% by weight of the makeup-removing composition.

Optionally, the makeup-removing composition consists of the at least one dimethicone, the second alkane, and optionally one or more excipients or adjuvants

Optionally, the makeup composition, the makeup-removing composition, or both, are anhydrous compositions.

Preferably, the makeup composition is substantially free of dimethicone.

Optionally, the makeup composition is substantially free of polyalkenes and/or surfactants. Optionally, the makeup composition and the makeup-removing composition are in separate plastic or glass jars, tubes, or bottles.

A second aspect of the present disclosure is drawn to a method for applying and removing makeup, comprising: applying a makeup composition to a keratin material (preferably eyelashes or skin around the eye), where the makeup composition is as described previously, and includes a styrenic block copolymer and an alkane solvent. After a period of time (which is typically a period of minutes or hours), a makeup-removing composition is applied, where the makeup-removing composition is as described previously, and includes an alkane solvent and a low viscosity dimethicone (each having a viscosity of 60,000 cst or less). After another period of time (preferably less than 60 seconds), the makeup composition and makeup-removing composition are removed from the keratin material.

A third aspect of the present disclosure is drawn to a method for forming a gel from a styrenic block copolymer on a keratin material. The method first includes applying a makeup-removing composition over a coated keratin material. The makeup-removing composition (e.g., a mascara-removing composition) is as described previously, and includes an alkane solvent and one or more low viscosity dimethicones (each having a viscosity of 60,000 cst or less). The coated keratin material should be coated with a makeup composition (such as a mascara) as described previously, including a styrenic block copolymer and an alkane solvent. The makeup-removing composition should remain in contact with the coated keratin material long enough to allow the styrenic block copolymer to precipitate and form a gel (typically 10-60 seconds). Optionally, the formed gel can then be removed from the keratin material.

### BRIEF DESCRIPTION OF DRAWINGS

FIG. 1 is a graph illustrating the viscosity of gels formed from combining (i) a styrenic block copolymer in an alkane solvent with (ii) various dimethicones, in 3:1 ratios.

FIG. 2 is a graph illustrating the elasticity of select combinations of various exemplary and comparative mascaras and mascara removing compositions.

FIG. 3 is a graph illustrating the critical strain of select combinations of various exemplary and comparative mascaras and mascara removing compositions.

FIG. 4 is a graph illustrating the zeroth shear viscosity of select combinations of various exemplary and comparative mascaras and mascara removing compositions.

### DETAILED DESCRIPTION

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “alkane” means a straight chain or branched non-cyclic saturated hydrocarbon that is not a gas at standard temperature and pressure, and preferably those alkanes with a boiling point (Tbp) greater than 80° C.

As used herein, the term “polymer” is intended to denote compounds comprising at least two repeating units, preferably at least three repeating units and especially at least 10 repeating units.

As used herein, the term “substantially free [of an ingredient]” means that the composition contains less than 1% of the identified ingredient.

All percentages listed are by weight unless otherwise noted.

As disclosed above, a first aspect of the present disclosure is drawn to a system or kit, that comprises, consists essentially of, or consists of the novel combination of specific makeup compositions and specific makeup-removing compositions. These makeup compositions and makeup-removing compositions may be provided to users in separate plastic or glass jars, tubes, or bottles.

### Makeup Compositions

The makeup compositions are preferably eye makeup compositions, and most preferably a mascara. In preferred embodiments, the makeup composition is an anhydrous composition.

The disclosed makeup compositions must contain at least two materials: (i) a styrenic block copolymer, and (ii) an alkane. The makeup composition may contain other components, including waxes, filler agents, additional polymers, additional solvents, and colorants. In some embodiments, the makeup composition consists essentially of, or consists of, the styrenic block copolymer, the alkane, optional waxes, optional filler agents, optional additional polymers, optional additional solvents, and optional colorants.

In some embodiments, the makeup composition is free, or substantially free, of additional polymers and/or additional solvents. In some embodiments, the makeup composition is free, or substantially free, of a surfactant. In some embodiments, the makeup composition is free, or substantially free, of dimethicone, polyalkenes, or both. In preferred embodiments, the makeup composition is substantially free of dimethicone. In more preferred embodiments, the makeup composition is free, or substantially free, of low viscosity dimethicones.

### Styrenic Block Copolymer

The makeup compositions will include one or more styrenic block copolymers. The styrenic block copolymer is generally a hydrocarbon-based block copolymer which is preferably soluble or dispersible in a fatty phase or mixture containing fatty substances. In the present invention, the fatty substances are chosen from oils and waxes. The styrenic block copolymer is capable of thickening or gelling the fatty phase or mixture containing fatty substances.

Preferably, the styrenic block copolymer is an amorphous polymer, which means a polymer that does not have a crystalline form. Such a compound has film-forming properties, i.e. it is capable of forming a film when applied to the skin.

Preferably, the styrenic block copolymer is obtained from at least one styrene monomer.

The styrenic block copolymer may especially be a diblock, triblock, multiblock, radial or star copolymer, or mixtures thereof.

Such styrenic block copolymer are described in patent application US-A-2002/005 562 and in U.S. Pat. No. 5,221,534, which are incorporated by reference herein in their entirety.

The copolymer may contain at least one block whose glass transition temperature is preferably less than 20° C., preferably less than or equal to 0° C., preferably less than or equal to −20° C. and more preferably less than or equal to −40° C. The glass transition temperature of the said block may be between −150° C. and 20° C. and especially between −100° C. and 0° C.

The styrenic block copolymer present in the composition according to the invention is an amorphous copolymer formed by polymerization of an olefin. The olefin may especially be an elastomeric ethylenically unsaturated monomer.

Examples of olefins that may be mentioned include ethylenic carbide monomers, especially containing one or two ethylenic unsaturations and containing from 2 to 5 carbon atoms, such as ethylene, propylene, butadiene, isoprene or pentadiene.

Advantageously, the styrenic block copolymer is an amorphous block copolymer of styrene and of olefin.

Block copolymers comprising at least one styrene block and at least one block comprising units chosen from butadiene, ethylene, propylene, butylene and isoprene or a mixture thereof are especially preferred. According to one preferred embodiment, the styrenic block copolymer is hydrogenated to reduce the residual ethylenic unsaturations after the polymerization of the monomers.

In particular, the styrenic block copolymer is a copolymer, optionally hydrogenated, containing styrene blocks and ethylene/C3-C4 alkylene blocks.

According to one preferred embodiment, the styrenic block copolymer comprises at least one diblock copolymer, which is preferably hydrogenated, preferably chosen from styrene-ethylene/propylene copolymers, styrene-ethylene butadiene copolymers and styrene-ethylene/butylene copolymers. The diblock polymers are especially sold under the name Kraton® GI 701 E by the company Kraton Polymers.

According to another preferred embodiment, the styrenic block copolymer comprises at least one triblock copolymer, which is preferably hydrogenated, preferably chosen from styrene-ethylene/propylene-styrene copolymers, styrene-ethylene/butadiene-styrene copolymers, styrene-isoprene-styrene copolymers and styrene-butadiene-styrene copolymers. Triblock polymers are especially sold under the names Kraton® G1650, Kraton® G1652, Kraton® G1657, Kraton® DI 101, Kraton® DI 102 and Kraton® DI 160 by the company Kraton Polymers.

According to one embodiment of the present invention, the at least one styrenic block copolymer is a diblock copolymer chosen from styrene-ethylene/butylene diblock copolymer, styrene-ethylene/propylene diblock copolymer, and mixtures thereof.

According to another embodiment of the present invention, the styrenic block copolymer is a styrene-ethylene/butylene-styrene triblock copolymer.

According to one preferred embodiment of the invention, the styrenic block copolymer is a mixture of a styrene-ethylene/butylene-styrene triblock copolymer and of a styrene-ethylene/butylene diblock copolymer, especially the products sold under the name Kraton® G1657M or Kraton® G1657MS by the company Kraton Polymers.

According to another preferred embodiment of the invention, the styrenic block copolymer is a mixture of styrene-butylene/ethylene-styrene hydrogenated triblock copolymer and of ethylene-propylene-styrene hydrogenated star polymer, such a mixture possibly being especially in isododecane or in another oil. Such mixtures are sold, for example, by the company Penreco under the trade names Versagel® M5960 and Versagel® M5670.

In particularly preferred embodiments of the present invention, the styrenic block copolymer is a mixture of styrene-ethylene/butylene-styrene triblock copolymer and styrene-ethylene/butylene diblock copolymer. Preferably, the percent amount of the triblock copolymer is greater than the percent amount of the diblock polymer in the mixture, based on the total weight of the mixture. For example, the mixture can contain 70% by weight of the triblock copolymer and 30% by weight of the diblock copolymer. Such a mixture is available by the INCI name hydrogenated styrene/butadiene copolymer, sold under the tradename Kraton® G1657M or Kraton® G1657MS by the company Kraton Polymers.

The content of styrenic block copolymer may range from about 0.2% to about 7% by weight, preferably from about 0.5% to about 6% by weight, more preferably from about 1% to about 5% by weight, based on the total weight of the makeup composition, including all ranges and subranges therebetween.

### Alkane

The makeup compositions will include at least one alkane as a solvent for the styrenic block copolymer. The alkane in the makeup composition may be any appropriate cosmetically-acceptable alkane. Preferably, the alkane has a carbon chain between 10 and 24 carbons, and more preferably having carbon chains containing between 12 and 18 carbons. Preferably, the alkane is isododecane, isohexadecane, or both.

In some embodiments, only a single alkane is present in the makeup composition. In other embodiments, two or more alkanes are present in the makeup composition.

In embodiments of the makeup composition, the content of the alkane in the makeup composition is no less than 10%, 20%, 30%, 40%, or 50%, and no more than 70%, 65%, or 60% by weight of the makeup composition, including all combinations of ranges and subranges therebetween. Preferably, the amount of alkane is between about 10% and about 70% by weight of the makeup composition, and more preferably between about 40% and about 60% by weight of the makeup composition.

### Waxes

The makeup composition may optionally contain a wax. The waxes preferably have a melting point greater than 35° C., such as from between greater than 35° C. to about 250° C. or such as from between about 40° C. to about 100° C. The waxes having a melting point greater than 35° C. is defined as having a reversible change of solid/liquid state. The melting point of a wax in solid form is the same as the freezing point of its liquid form, and depends on such factors as the purity of the substance and the surrounding pressure. The melting point is the temperature at which a solid and its liquid are in equilibrium at any fixed pressure. A solid wax begins to soften at a temperature close to the melting point of the wax. With increasing temperature, the wax continues to soften/melt until at a particular temperature, the wax completely becomes liquid at a standard atmospheric pressure. It is at this stage that an actual melting point value is given for the material under consideration. When heat is removed, the liquefied wax material begins to solidify until the material is back in solid form. By bringing the wax material to the liquid state (melting), it is possible to make it miscible with other materials such as oils, and to form a microscopically homogeneous mixture. However, when the temperature of the mixture is brought to room temperature, recrystallization of the wax with the other materials in the mixture may be obtained.

The melting points of the wax (e) s and the particles of the aqueous dispersion of the present disclosure may be determined according to known methods or apparatus such as by differential scanning calorimetry, Banc Koffler device, melting point apparatus, and slip melting point measurements.

The melting point of the wax(es) may also be defined as the temperature at which the peak endothermic heat flow occurs in a differential scanning calorimetry sweep.

The wax(es) which may be present in the particles of the present disclosure and have a melting point of greater than 35° C. is chosen from waxes that are solid or semisolid at room temperature.

The wax(es) which may be present in the particles of the present disclosure may be chosen from waxes that have hardness values ranging from about 0.001 MPa (Mega Pa) to about 15 MPa, or such as from about 1 MPa to about 12 MPa, or such as from about 3 MPa to about 10 MPa.

The hardness of the wax may be determined by any known method or apparatus such as by needle penetration or using the durometer or texturometer.

Natural waxes include animal, vegetable/plant, mineral, or petroleum derived waxes. They are typically esters of fatty acids and long chain alcohols. Wax esters are derived from a variety of carboxylic acids and a variety of fatty alcohols. The waxes that may comprise the particle of the present disclosure may also be known as solid lipids.

Examples of suitable waxes include, but are not limited to, beeswax, carnauba wax, candelilla wax, ouricoury wax, Japan wax, cork fibre wax or sugar cane wax, rice or rice bran wax, montan wax, paraffin wax, lignite wax or microcrystalline wax, and ozokerite.

Particularly preferred waxes having a melting point of greater than 35° C. are beeswax, carnauba wax, candelilla wax, paraffin wax, and/or rice bran wax.

The wax(es) which may be present in the particles of the present disclosure may be chosen from soft waxes and from hard waxes. Soft waxes may be defined as those waxes which have a melting point of below about 70° C., and preferably, a melting point of below about 60° C. Hard waxes may be defined as those waxes which have a melting point of equal to or greater than about 70° C., and preferably, a melting point of equal to or greater than about 60° C.

According to one embodiment, soft waxes according to the present disclosure include, but are not limited to, paraffin wax, stearic alcohol, ozokerite, synthetic beeswax, beeswax, and candelilla wax.

According to one embodiment, hard waxes according to the present disclosure, include, but are not limited to, carnauba wax, microcrystalline wax, polyethylene wax, and rice bran wax.

In some embodiments, the wax may be employed in an amount ranging from about 5%, about 10%, or about 15%, to about 20%, about 25%, about 30%, about 40%, about 50%, or about 60% by weight, or preferably from about 10% to about 40% by weight, such as from about 15% to about 30% by weight, or such as from about 15% to about 25% by weight of the makeup composition, including all combinations of ranges and subranges therebetween.

### Filler Agents

The makeup compositions may optionally include at least one filler. As used herein, the term “filler” means any particle that is solid at room temperature and atmospheric pressure, used alone or in combination, which does not react chemically with the various ingredients of the emulsion and which is insoluble in these ingredients, even when these ingredients are raised to a temperature above room temperature and in particular to their softening point or their melting point. In an embodiment, the at least one filler has a melting point at least greater than 1700° C., for example, greater than 2000° C. In an embodiment, the at least one filler may have an apparent diameter ranging from 0.01 μm to 150 μm, such as from 0.5 μm to 120 μm, for example from 1 μm to 80 μm. An apparent diameter corresponds to the diameter of the circle into which the elementary particle fits along its shortest dimension (thickness for leaflets). Further, the at least one filler may be absorbent, i.e., capable in particular of absorbing the oils of the composition and also the biological substances secreted by the skin, may be surface-treated, e.g., to make it lipophilic, and/or may be porous so as to absorb the sweat and/or sebum secreted by the skin.

The one or more fillers may be chosen from inorganic and organic fillers, and may have any shape such as lamellar, spherical and/or oblong. Non-limiting examples of the at least one inert filler include talc, mica, silica, and disteardimonium hectorite.

In some embodiments, when present, the one or more fillers are present in an amount less than 10% by weight of the makeup composition, such as at least 1%, 2%, 3%, 4%, or 5%, and no more than 10%, 9%, 8%, 7%, or 6%, and 10% by weight of the makeup composition, including all combinations of ranges and subranges therebetween.

### Additional Polymers

The makeup composition may contain one or more additional polymers. In some embodiments, the makeup composition contains two or more, three or more, four or more, or five or more additional polymers.

The additional polymers may include a lipophilic polyamide polymer. Mention may be made of polyamides branched with pendant fatty chains and/or terminal fatty chains containing from 12 to 120 carbon atoms and in particular from 12 to 68 carbon atoms, the terminal fatty chains being bonded to the polyamide backbone via ester groups. These polymers are more especially those described in document U.S. Pat. No. 5,783,657 from the company Union Camp. In particular, mention may be made of the polymers of which the INCI name is “ethylenediamine/stearyl dimer dilinoleate copolymer” and “ethylenediamine/stearyl dimertallate copolymer”.

The additional polymers may include copolymers of polyols and diacid dimers and esters thereof, such as Hailuscent ISDA or dilinoleic acid/butanediol copolymers.

The additional polymers may include film-forming polymers, which are compatible with the oil/wax phase and which forms a film after application to the lips. Suitable polymers include homo- and copolymers of polyvinylpyrrolidone (PVP) and vinyl pyrrolidone (VP), e.g., vinyl pyrrolidone (VP)/hexadecene copolymer, PVP/hexadecene copolymer (an alkylated polyvinyl pyrrolidone copolymer), and VP/eicosene copolymer, resin MK (polymethylsilsesquioxane), silicone acrylates (e.g., KP 550 from Shin-Etsu) and acrylates copolymer. Other examples of film formers include vinylpyrrolidone/vinyl acetate (PVP/VA) copolymers such as the Luviskol VA grades (all ranges) from BASF® Corporation, and the PVP/VA series from ISP; acrylic fluorinated emulsion film formers, including Foraperle® film formers, such as Foraperle® 303 D from Elf Atochem; GANEX® copolymers, such as Butylated PVP, PVP/Hexadecene copolymer, PVP/Eicosene copolymer or tricontanyl; Poly (vinylpyrrolidone/diethylaminoethyl methacrylate) or PVP/Dimethylaminoethylmethacrylate copolymers such as Copolymer 845; Resin ACO-5014 (Imidized IB/MA copolymer); other PVP based polymers and copolymers. Film formers also include silicone gums; cyclomethicone and dimethicone crosspolymers (For example, Dow Corning® 2-9040, See U.S. Pat. No. 5,654,362, the disclosure of which is hereby incorporated by reference); trimethyl siloxysilicate, such as SR 1000, SS4230, or SS4267 available from GE Silicones; alkyl cycloalkylacrylate copolymers (See WO98/42298 the disclosure of which is hereby incorporated by reference); or Mexomere® film formers and other allyl stearate/vinyl acetate copolymers (allyl stearate/VA copolymers). Film formers also include polyolprepolymers such as PPG-12/SMDI copolymer, polyolprepolymers such as PPG-12/SMDI copolymer, poly(oxy-1,2-ethanediyl), alpha-hydro-omega-hydroxy-polymer with 1,1′-methylene-bis-(4-isocyanatocyclohexane) available from Barnet; Avalure™ AC Polymers (Acrylates Copolymer) and Avalure™ UR polymers (Polyurethane Dispersions), available from BFGoodrich. Further examples of film formers include polyvinyl stearate, polyvinyl stearate crosslinked with the aid of divinylbenzene, of diallyl ether or of diallyl phthalate copolymers, polystearyl (meth)acrylate, polyvinyl laurate and polylauryl (meth)acrylate copolymers, it being possible for these poly(meth)acrylates to be crosslinked with the aid of ethylene glycol dimethacrylate or tetraethylene glycol dimethacrylate.

In some embodiments, when present, the one or more additional polymers are present in an amount less than 20% by weight of the makeup composition, such as at least 1%, 3%, 5%, 7%, 9%, or 11%, and no more than 20%, 18%, 16%, or 14%, by weight of the makeup composition, including all combinations of ranges and subranges therebetween.

### Additional Solvents

The makeup composition may contain one or more additional solvents, in addition to the alkane. In some embodiments, the makeup composition may include only two additional solvents. In some embodiments, the makeup composition may include two or more additional solvents.

The additional solvent may include one or more solvents, including volatile organic solvents such as C2 to C4 mono-alcohols (such as ethanol, isopropyl alcohol, butanol), aromatic alcohols (such as benzyl alcohol, phenylethanol), polyols such as C2-C6 glycols (such as propylene glycol, butylene glycol, pentylene glycol, hexylene glycol, glycerol), volatile polyol ethers, volatile glycol ethers, acetone, and carbonate esters (such as propylene carbonate), and mixtures thereof.

In some embodiments, when present, the one or more additional solvents are present in an amount less than 5% by weight of the makeup composition, such as at least 0.1%, 0.5%, 1%, or 1.5%, and no more than 5%, 4%, 3%, 2.5%, or 2% by weight of the makeup composition, including all combinations of ranges and subranges therebetween.

### Optional Excipients or Adjuvants

In some embodiments, the makeup composition may include, e.g., preservatives, surfactants, thickeners, moisturizing agents, chelators, buffers, essential oils, neutralizing or pH adjusting agents, fragrances, antifoaming agents, wetting agents, antioxidants, etc. These optional excipients or adjuvants are typically used in conventional amounts, such as up to about 20%, 10%, or 5% by weight of the finished formulation.

### Colorants

The makeup composition will preferably contain at least one colorant. Colorants are typically chosen from the lipophilic dyes, hydrophilic dyes, traditional pigments, and nacres usually used in cosmetic or dermatological compositions, and mixtures thereof. The colorant may have any shape, such as, for example, spheroidal, oval, platelet, irregular, and mixtures thereof. Pigments may optionally be surface-treated e.g., with silicones, perfluorinated compounds, lecithin, and amino acids.

The liposoluble dyes include, for example, Sudan Red, D&C Red 17, D&C Green 6, β-carotene, soybean oil, Sudan Brown, D&C Yellow 11, D&C Violet 2, D&C Orange 5 and quinoline yellow.

The pigments may be chosen from white pigments, colored pigments, inorganic pigments, organic pigments, coated pigments, uncoated pigments, pigments having a micron size and pigments not having a micron size. Among the inorganic pigments that may be mentioned are titanium dioxide, zirconium oxide, zinc oxide, cerium oxide, chromium oxide, manganese violet, ultramarine blue, chromium hydrate, and ferric blue. Among the organic pigments which may be mentioned are carbon black, pigments of D&C type, lakes based on cochineal carmine, lakes based on barium, lakes based on strontium, lakes based on calcium, and lakes based on aluminum.

The nacreous pigments may, for example, be chosen from white nacreous pigments such as mica coated with titanium and mica coated with bismuth oxychloride, colored nacreous pigments such as titanium mica with iron oxides, titanium mica with, for example, ferric blue and/or chromium oxide, titanium mica with an organic pigment of the type mentioned above, as well as nacreous pigments based on bismuth oxychloride, interferential pigments, and goniochromatic pigments.

The pigments can also be spherical scattering agents such as spherical powders that achieve a soft focus look. Examples include calcium aluminum borosilicate, PMMA, polyethylene, polystyrene, methyl methacrylate crosspolymer, nylon-12, ethylene/acrylic acid copolymer, boron nitride, Teflon, or silica.

Colorants can generally be present in an amount ranging from about 0.01% to about 30% relative to the total weight of the composition. In some embodiments, the colorants are present in an amount no less than 0.1% by weight and more than 10%, 9%, 8%, 7%, 6%, or 5% by weight of the makeup composition, including all combinations of ranges and subranges therebetween.

### Makeup-Removing Compositions

The makeup-removing composition is intended to allow the makeup compositions disclosed above to be readily removed from a keratin surface. The makeup-removing composition must contain an alkane and one or more low viscosity dimethicones (each having a viscosity of 60,000 cst or less). In some embodiments, the makeup-removing composition is a mascara-removing composition (and the makeup composition is a mascara).

In some embodiments, the makeup-removing composition is free, or substantially free, of water. In some embodiments, the makeup composition, the makeup-removing composition, or both, are anhydrous compositions.

In some embodiments, the makeup-removing composition consists essentially of, or consists of, the alkane and the one or more low viscosity dimethicones. In some embodiments, the makeup-removing composition comprises the alkane and the one or more low viscosity dimethicones. In some embodiments, the makeup-removing composition consists of, or consists essentially of, the at least one dimethicone, the second alkane, and optionally one or more excipients or adjuvants.

### Alkane

The makeup-removing compositions will include at least one alkane that can act as a solvent for the styrenic block copolymer when the makeup-removing composition is applied over the makeup composition. The alkane in the makeup-removing composition may be any appropriate cosmetically-acceptable alkane. Preferably, the alkane has a carbon chain between 10 and 24 carbons, and more preferably having carbon chains containing between 12 and 18 carbons. Preferably, the alkane is isododecane, isohexadecane, or both.

In some embodiments, only a single alkane is present in the makeup-removing composition. In other embodiments, two or more alkanes are present in the makeup-removing composition.

In embodiments of the makeup-removing composition, the content of the alkane in the makeup composition is no less than 25%, 30%, 40%, 50%, 60%, 70%, or 80%, and no more than 95%, 90%, 80%, or 70% by weight of the makeup-removing composition, including all combinations of ranges and subranges therebetween. Preferably, the amount of alkane is between about 30% and about 90% by weight of the makeup-removing composition. In some embodiments, the alkane is present in an amount between about 60% and about 90% by weight of the makeup-removing composition.

In some embodiments, the alkane in the makeup composition and the alkane in the makeup-removing composition are the same alkane. In some embodiments, the alkane in the makeup composition and the alkane in the makeup-removing composition are different alkanes.

In some embodiments, all of the alkanes in the composition have between 12 and 18 carbons.

### Low Viscosity Dimethicones

The makeup-removing composition must include a low-viscosity dimethicone (polydimethylsiloxane, PDMS). Low-viscosity dimethicones have viscosities less than 60,000 centistokes (cst) when measured at 25° C. Preferably, the viscosity of the low-viscosity dimethicone is between 5 cst and 60,000 cst.

In embodiments of the makeup-removing composition, the viscosity of each low viscosity the dimethicone is no less than 5 cst, 10 cst, 50 cst, 100 cst, 350 cst, or 1,000 cst, and no more than 60,000 cst, 30,000 cst, 10,000 cst, 5,000 cst, 4,000 cst, 3,000 cst, 2,000 cst, 1,500 cst, or 1,000 cst, including all combinations of ranges and subranges therebetween.

In some embodiments, the makeup-removing composition is free, or substantially free, from dimethicones other than low viscosity dimethicones.

The weighted average viscosity of all dimethicones combined should not exceed 60,000 cst. That is, if the formula contains 30% of dimethicone A (with a viscosity of 30,000 cst) and 40% of dimethcone B (with a viscosity of 100,000 cst), the weighted average is 70,000 ([30%×30,000+40%×100,000]/70%=70,000) and would exceed that 60,000 cst target. Preferably, the weighted average viscosity of all dimethicones combined should not exceed 30,000 cst, more preferably, the weighted average viscosity of all dimethicones combined should be between not exceed 10,000 cst, and still more preferably, the weighted average viscosity of all dimethicones combined should not exceed 5,000 cst,

In embodiments of the makeup-removing composition, the total content of all low viscosity dimethicones in the makeup composition is no less than 5%, 10%, 15%, 20%, 30%, 40%, 50%, or 60%, and no more than 70%, 60%, 50%, 40%, 30%, 20%, or 15% by weight of the makeup-removing composition, including all combinations of ranges and subranges therebetween. Preferably, the total amount of low viscosity dimethicones is between about 10% and about 70% by weight of the makeup-removing composition. In some embodiments, the total amount of low viscosity dimethicones is between about 5% and about 15% by weight of the makeup-removing composition.

### Optional Excipients or Adjuvants

In some embodiments, the makeup composition may include, e.g., preservatives, surfactants, thickeners, moisturizing agents, chelators, buffers, essential oils, neutralizing or pH adjusting agents, fragrances, antifoaming agents, wetting agents, antioxidants, etc. These optional excipients or adjuvants are typically used in conventional amounts, such as up to about 20%, 10%, or 5% by weight of the finished formulation.

A second aspect is drawn to a method for applying and removing makeup. The method involves first applying a makeup composition (such as a mascara) to a keratin material (preferably eyelashes or skin around the eye), where the makeup composition is as described previously. Specifically, the makeup composition includes a styrenic block copolymer and an alkane solvent.

After a period of time, where the makeup is worn by the user, typically for minutes or hours (such as roughly 4-12 hours), a makeup-removing composition (such as a mascara-removing composition) is applied. The makeup-removing composition is as described previously. Specifically, the makeup-removing composition includes an alkane solvent and a low viscosity dimethicone (each having a viscosity of 60,000 cst or less).

After another period of time, the makeup composition and makeup-removing composition are removed from the keratin material. That period of time should be less than 60 seconds. In some embodiments, it is between 10 and 30 seconds, or between 10 and 60 seconds.

A third aspect is drawn to a method for forming a gel from a styrenic block copolymer on a keratin material. The method first includes applying a makeup-removing composition over a coated keratin material.

The coated keratin material should be a keratin material that had been previously coated with a makeup composition as described previously. Specifically, a makeup composition should have previously been applied to the keratin material, where the makeup composition includes a styrenic block copolymer and an alkane solvent.

The makeup-removing composition is as described previously, and specifically is a composition that includes an alkane solvent and one or more low viscosity dimethicones (each having a viscosity of 60,000 cst or less).

After the makeup-removing composition is applied, it should remain in contact with the coated keratin material long enough to allow the styrenic block copolymer to precipitate and form a gel. This is preferably less than 60 seconds, and typically between 10 and 60 seconds.

After this time period, the formed gel can then be removed from the keratin material, which can generally be accomplished using, e.g., a wipe, cotton pad, or cloth.

### Example 1

It is disclosed that the physical gelling of a styrenic block copolymer incorporated into simple mascara composition (in this example, hydrogenated styrene/butadiene copolymer in isododecane was used) relies on choosing the right viscosity of the dimethicone in the remover. The gelling interaction between blends of the styrenic block copolymer and dimethicone increase the cohesiveness of mascara which aids in easy removal.

To illustrate this, zeroth shear viscosity can be measured. The styrenic block copolymer and a dimethicone was blended at 3 to 1 ratio, using dimethicones with various viscosities (from 5 cst to 100,000 cst).

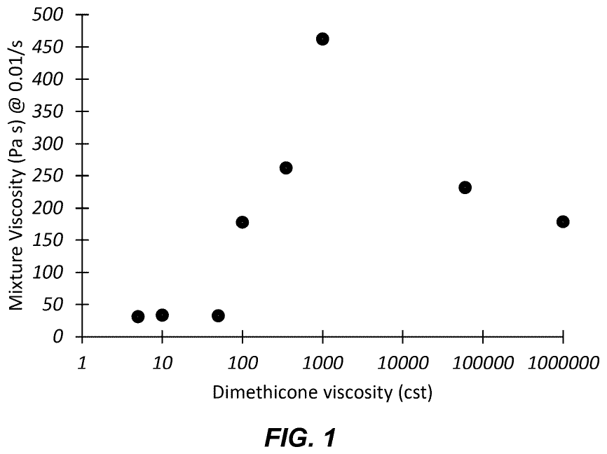

The combination was first deposited onto the rheology bottom plate. A 20 mm 2-degree cone plate was used as a rheology probe with a gap of 57 μm between the bottom plate and the probe. Each sample was first equilibrated at 25° C. for 20 seconds, preshear using 10/s for 30 seconds, then a shear rate flow experiment was performed. The duration of each experiment was 10 minutes, shear rate changes from 0.001-200/s, with 5 data points recorded within each decade. After each experiment, a zero-shear viscosity was determined from a log (viscosity) vs. log (shear rate) plot, by linear fitting the initial plateau region to intersect with y-axis. This value represents the viscosity of each mixture under unperturbed situation. The resulting values can be seen in FIG. 1. By comparing the viscosity of the styrenic block copolymer/isododecane/dimethicone mixture vs. dimethicone, a percentage increase was calculated and listed in Table 1, below. It can be seen that dimethicones with viscosity less than 60,000 cst appear suitable for gelling the styrenic block copolymer, and thus for easy removal.

TABLE 1
Mixture Viscosity as a Percentage
Increase over Dimethicone

| | Mixture |
|---|---|
| | Viscosity % |
| Dimethicone | Increase vs. |
| (cst) | Dimethicone |
| 5 | 619,000 |
| 10 | 332,700 |
| 50 | 64,600 |
| 100 | 177,785 |
| 350 | 74,879 |
| 1000 | 46,236 |
| 60000 | 386 |
| 1000000 | 18 |

Further, when adding the mascara-removing composition to the mascara composition, a short gelling time is generally preferred. Based on previously described zeroth shear viscosity assessment, 1000 cst dimethicone was chosen to perform a gelling time study. A rheometer is used to characterize the physical curing time of the second step to the first step. In this example, 0.6 g of the styrenic block copolymer in isododecane is first deposited onto the bottom plate, and 0.4 g of dimethicone 1000 cst is deposited on top of that. A 40 mm flat plate is used as a rheology probe with a gap of 1000 μm between the bottom plate and the probe. Time-sweep is performed by using strain=0.1%, and angular frequency=1 rad/s. Curing time is determined by the time which the two compositions reach equilibrium viscosity, which can be seen graphically, e.g., when the loss modulus is equal to the storage modulus as measured by the rheometer. Here, the curing time for the styrenic block copolymer/isododecane/dimethicone 1000 cst is 11.7 seconds.

### Example 2—Makeup Compositions and Makeup-Removing Compositions

Seven mascara compositions were created, where the levels of the styrenic block copolymer (here, hydrogenated styrene/butadiene copolymer) and alkane solvent (isododecane) were varied, while the five waxes, two fillers, five additional polymers, two additional solvents, and two pigments were kept constant across all formulations. See Table 2, below. There were five exemplary mascaras (M1-M5), where the styrenic block copolymer is present in amounts between 0.2% and 7%, and two comparative mascaras (CM1-CM2) where the styrenic block copolymer was not present or present at 10%. To produce each formulation, all raw materials were combined in a closed kettle, heated up to 90° C., and mixed until homogenous, after which the formulation was cooled down to room temperature and poured into an appropriate container.

TABLE 2

| Material | M1 | M2 | M3 | M4 | M5 | CM1 | CM2 |
|---|---|---|---|---|---|---|---|
| Hydrogenated | 0.40% | 1% | 2% | 5% | 7% | 0 | 10% |
| Styrene/Butadiene | | | | | | | |
| Copolymer | | | | | | | |
| Isododecane | 45-75% | 45-75% | 45-75% | 45-75% | 45-75% | 45-75% | 45-75% |
| Waxes (5) | 15-20% | 15-20% | 15-20% | 15-20% | 15-20% | 15-20% | 15-20% |
| Fillers (2) | 5-10% | 5-10% | 5-10% | 5-10% | 5-10% | 5-10% | 5-10% |
| Additional | 5-15% | 5-15% | 5-15% | 5-15% | 5-15% | 5-15% | 5-15% |
| Polymers (5) | | | | | | | |
| Additional | 1-5% | 1-5% | 1-5% | 1-5% | 1-5% | 1-5% | 1-5% |
| Solvents (2) | | | | | | | |
| Pigments (2) | 1-5% | 1-5% | 1-5% | 1-5% | 1-5% | 1-5% | 1-5% |

Nine mascara-removing compositions were also created, using the alkane solvent and two low viscosity dimethicones with different viscosities. See Table 3, below. The nine mascara-removing compositions included six exemplary mascara-removing compositions (MR1-MR6) where the composition included one of the two dimethcones in amounts between 10% and 70%, with isododecane as the alkane solvent, and three comparatives (CMR1-CMR3), where the composition included only a single material-either one of the two dimethicones, or the isododecane. To produce each formulation, all raw materials were combined at room temperature, mixed until homogenous, and poured into an appropriate container.

TABLE 3

| Material | MR1 | MR2 | MR3 | MR4 | MR5 | MR6 | CMR1 | CMR2 | CMR3 |
|---|---|---|---|---|---|---|---|---|---|
| Dimethicone | 10% | — | 70% | — | 50% | — | 100% | — | — |
| 5 cst | | | | | | | | | |
| Dimethicone | — | 10% | — | 70% | — | 50% | — | 100% | — |
| 1000 cst | | | | | | | | | |
| Isododecane | 90% | 90% | 30% | 30% | 50% | 50% | — | — | 100% |

### Evaluations

The various combinations of makeup and makeup-removers was evaluated for, e.g., removability, elasticity, critical strain, and zeroth shear viscosity.

Each mascara formula (M1-M5, CM1-CM2) was first casted into a film on a drawdown paper using 1 ml drawdown bar, and air dried under room temperature overnight. One drop of a removing solution was placed onto three separate locations of the dried mascara film, after which a period of time (of 10 s, 30 s and 60 s, respectively) was given for each drop to interact with the dried mascara film. A kimwipe was folded twice for removing, and each removing solution drop was wiped 5 times and the remaining film is checked. The removability score was rated based on residue of mascara on the drawdown card, from 0 (not removed)-5 (most removed). Tables 4-6, below, shows a summary of some of the data.

TABLE 4
10 Second Removal Evaluation

| | CMR3 | MR1 | MR2 | MR3 | MR4 | MR5 | MR6 |
|---|---|---|---|---|---|---|---|
| CM1 | 0.5 | 1.5 | 0.5 | 1.25 | 1.75 | 1 | 1 |
| M1 | 0.5 | 1.5 | 0.5 | 1.25 | 1.75 | 1.5 | 1.5 |
| M2 | 0.5 | 1 | 2 | 0 | 0 | 0 | 0 |
| M3 | 2 | 4.5 | 4 | 1.5 | 0 | 1 | 3.75 |
| M4 | 2 | 3.5 | 1 | 0.5 | 0 | 0 | 0 |
| M5 | 1 | 0 | 1 | 0 | 0 | 1 | 1 |
| CM2 | 0 | 0 | 0 | 0 | 0 | 0 | 0 |

TABLE 5
30 Second Removal Evaluation

| | CMR3 | MR1 | MR2 | MR3 | MR4 | MR5 | MR6 |
|---|---|---|---|---|---|---|---|
| CM1 | 3 | 3.75 | 3.5 | 1.5 | 1 | 1 | 2 |
| M1 | 1.5 | 4.25 | 3.5 | 1.25 | 2.5 | 3.5 | 4.5 |
| M2 | 1.5 | 1.5 | 3 | 0 | 0 | 1.5 | 0.5 |
| M3 | 2 | 4.5 | 4.75 | 2.5 | 0.5 | 3 | 5 |
| M4 | 3.5 | 3.5 | 3 | 0.5 | 0 | 0 | 0.5 |
| M5 | 1 | 0 | 2 | 0 | 0 | 2 | 1.25 |
| CM2 | 0 | 0 | 0 | 0 | 0 | 0 | 0 |

TABLE 6
60 Second Removal Evaluation

| | CMR3 | MR1 | MR2 | MR3 | MR4 | MR5 | MR6 |
|---|---|---|---|---|---|---|---|
| CM1 | 3 | 4 | 3.5 | 3.5 | 1.5 | 2 | 3 |
| M1 | 3.25 | 4 | 4 | 1.5 | 3.5 | 5 | 5 |
| M2 | 3 | 3.25 | 3.25 | 0.5 | 0 | 1.5 | 1.5 |
| M3 | 3 | 4.75 | 5 | 2.5 | 0.5 | 4.25 | 5 |
| M4 | 4.5 | 3.75 | 4.75 | 0.5 | 1 | 0 | 0.5 |
| M5 | 1.25 | 0 | 4.5 | 0 | 0 | 1.5 | 1.5 |
| CM2 | 0 | 0 | 0 | 0 | 0 | 0 | 0 |

Surprisingly, the mascara was found to be far more removable when combinations of alkane and dimethicone as disclosed were used, with differences being apparent, in some cases, after only 10 seconds.

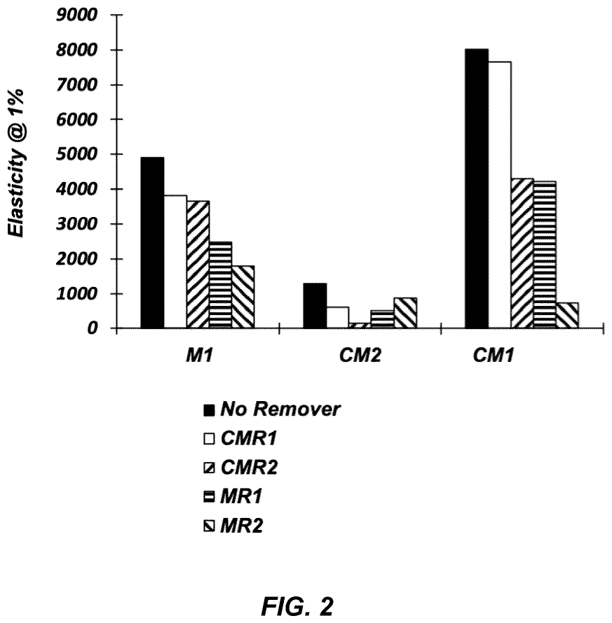

To determine elasticity, the inventive and comparative examples are mixed at a 75:25 ratio (mascara:mascara-remover) first. The elasticity of each composition was evaluated using rheology at 25° C., angular frequency=1 rad/s, where the elasticity was determined as the reading of the storage modulus at an oscillating strain of 1%. The results for some combinations can be seen in FIG. 2.

As can be seen in FIG. 2, for the novel makeup compositions (e.g., M1), the use of makeup removing compositions that use alkane and dimethicone mixtures (e.g., MR1, MR2) result in lower elasticities than makeup removing compositions that use dimethicone alone (e.g., CMR1, CMR2). Further, for the novel makeup compositions, use of 1000 cst dimethicone (e.g., CMR2, MR2) resulted in lower elasticities than 5 cst dimethicone (e.g., CMR1, MR1). That trend is also seen when no styrenic block copolymer is present in the mascara composition (e.g., CM1), although the differences between the elasticities using 5 cst and 1000 cst dimethicone are substantially increased.

However, surprisingly, with styrenic block copolymer at too high of an amount (here, CM2 has 10% styrenic block copolymer), the data is mixed—the elasticities using 5 cst dimethicone were lower than the elasticities using 1000 cst dimethicone, implying a different mechanism is at work, and the novel makeups and makeup-removing compositions will not work as disclosed when the level of styrenic block copolymer is above about 7% by weight of the makeup composition.

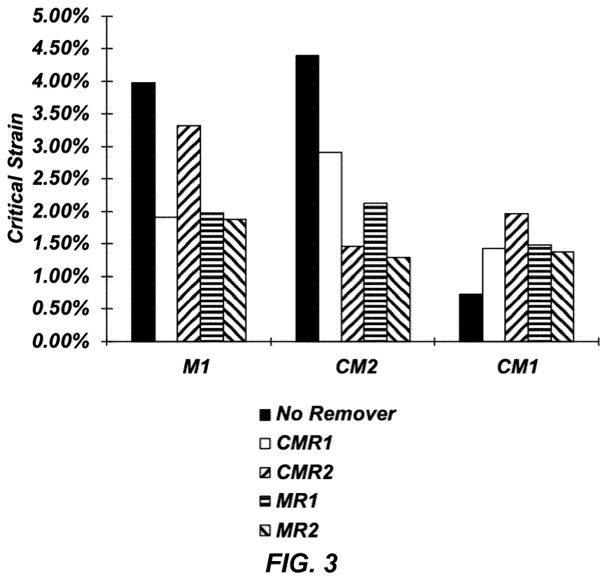

To determine critical strain, the inventive and comparative examples are mixed at a 75:25 ratio (mascara:mascara-remover) first. Critical strain of each composition is evaluated using rheology at 25 C, angular frequency=1 rad/s, where the critical strain value is determined from the region at which the elastic modulus or storage modulus G′ begins to be dependent on oscillation strain. The results for some combinations can be seen in FIG. 3.

As seen in FIG. 3, the use of even a small amount of styrenic block copolymer increases the critical strain of the formulation—as expected, CM1 by itself (with no styrenic block copolymer) has a very low critical strain as compared to M1 and CM2.

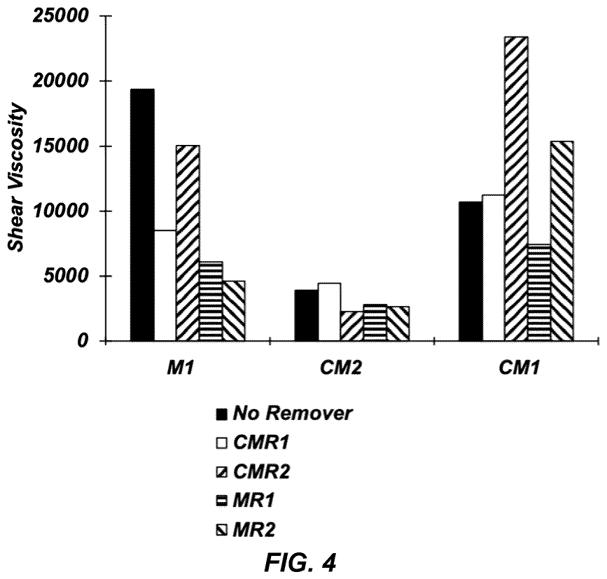

To determine zeroth shear viscosity, approximately 1 gram of each combination of exemplary and comparative mascara and mascara-removing compositions were deposited onto the bottom plate. A 40 mm flat plate is used as a rheology probe with a gap of 1000 μm between the bottom plate and the probe. Each sample was first equilibrated at 25° C. for 20 seconds, then a shear rate flow experiment was performed. The duration of experiment is 10 minutes, shear rate changes from 0.001-1000/s, with 5 data points recorded within each decade. After the experiment, a zero-shear viscosity is determined from a log (viscosity) vs. log (shear rate) plot, by linear fitting the initial plateau region to intersect with y-axis. This value represents the viscosity of each formula under unperturbed situation. The zeroth shear viscosities for some combinations can be seen in FIG. 4.

Those skilled in the art will recognize or be able to ascertain using no more than routine experimentation many equivalents to the specific embodiments of the invention described herein. Such equivalents are intended to be encompassed by the following claims.

What is claimed is:

1. A method for forming a gel on a keratin material, comprising:
providing a keratin material coated with a first composition comprising a styrenic block copolymer and a first alkane;
applying a second composition over the coated keratin material, the second composition comprising at least one low viscosity dimethicone and a second alkane, each low viscosity dimethicone having a viscosity of 60,000 cst or less;
after applying the second composition, allowing the styrenic block copolymer to precipitate and form a gel; and
after a period of time, removing at least the gel from the keratin material.

2. The method of claim 1, wherein the period of time is between 10 seconds and 60 seconds.

3. The method of claim 1, wherein the first alkane and the second alkane comprise different alkanes.

4. The method of claim 1, wherein the first alkane and the second alkane comprise the same alkane.

5. The method of claim 1, wherein the first alkane and the second alkane each have between 12 and 18 carbons.

6. The method of claim 1, wherein the first alkane is present in an amount of between 10% and 70% by weight of the first composition.

7. The method of claim 1, wherein the second alkane is present in an amount of between 30% and 90% by weight of the second composition.

8. The method of claim 1, wherein the styrenic block copolymer is present in an amount of between 0.2% and 7.0% by weight of the first composition.

9. The method of claim 8, wherein the styrenic block copolymer is a hydrogenated styrene/butadiene copolymer.

10. The method of claim 1, wherein the at least one low viscosity dimethicone is present in an amount of between 10% and 70% by weight of the second composition.

11. The method of claim 1, wherein the second composition consists of the at least one low viscosity dimethicone, the second alkane, and optionally one or more excipients or adjuvants.

12. The method of claim 1, wherein the first composition, the second composition, or both, are anhydrous compositions.

13. The method of claim 1, wherein the first composition is substantially free of dimethicone.

14. The method of claim 1, wherein the first composition is substantially free of polyalkenes.

15. The method of claim 1, wherein the first composition and the second composition are in separate plastic or glass jars, tubes, or bottles.

16. The method of claim 1, wherein the keratin material is an eyelash.
